(19) United States
(12) Patent Application Publication
(10) Pub. No.: US 2023/0263770 A1
(43) Pub. Date: Aug. 24, 2023

(54) THERAPY OF POST-OPERATIVE NAUSEA AND VOMITING

(71) Applicant: ACACIA PHARMA LTD, Duxford, Cambridge (GB)

(72) Inventors: Julian Clive GILBERT, Duxford (GB); Gabriel FOX, Duxford (GB); Robert William GRISTWOOD, Duxford (GB)

(73) Assignee: ACACIA PHARMA LTD, Duxford, Cambridge (GB)

(21) Appl. No.: 18/014,545

(22) PCT Filed: Jul. 6, 2021
(86) PCT No.: PCT/GB2021/051720

§ 371 Date: Jan. 5, 2023

(30) Foreign Application Priority Data
Jul. 6, 2020 (GB) 2010361.0
Jun. 9, 2021 (GB) 2108238.3

(51) Int. Cl. A61K 31/40; A61K 45/06; A61P 1/08

(52) U.S. Cl. CPC A61K 31/40; A61K 45/06; A61P 1/08; A61K 9/0019

(57) ABSTRACT

Amisulpride is useful in the therapy (particularly the prevention of) post-operative nausea and/or vomiting (PONV) in a patient, particularly wherein the patient has a high (≥30) BMI and/or the patient is a bariatric surgery patient.

### FIELD OF THE INVENTION

This invention relates to the therapy of post-operative nausea and/or vomiting (PONV). In particular, to the therapy of PONV in patients with a high BMI, i.e. a BMI of equal to or greater than about 30. The invention also relates to the therapy of PONV in patients undergoing bariatric surgery.

### BACKGROUND OF THE INVENTION

PONV is a condition that occurs in approximately 30% of all surgical patients and 70% of high-risk patients. Risk factors for PONV include: type of surgery, sex, smoking history, prior history of PONV or motion sickness, length of surgery, use of volatile anaesthetics and opioid analgesic usage. Typically, women are more prone than men to PONV, as are non-smokers and those who have previously experienced PONV or motion sickness.

PONV is a significant issue for patients and healthcare providers. It is often rated above postoperative pain as a complication most feared by patients and thus contributes significantly to anxiety and patient distress. PONV can delay discharge of the patient from hospital or result in readmission after inpatient procedures and can require admission for ambulatory patients. This has a significant economic and social impact. With increasing rates of hospital acquired resistant infections, it may also translate into an impact on clinical outcomes.

Numerous mechanisms have been implicated in PONV, most notably release of serotonin from the gut wall and activation of the chemoreceptor trigger zone in the brain. Consequently, several different receptors seem to be involved in PONV and represent effective targets for drug therapies. Among the most important are the serotoninergic 5HT₃ and the dopaminergic D₂ and possibly D₃ receptors.

Despite routine use of prophylactic anti-emetics in moderate and high-risk patients, PONV still occurs in about 30-40% of cases, even in patients receiving current standard-of care of 5HT₃ antagonists and corticosteroids and there remains a significant need for effective and safe additional agents, especially with different mechanisms of action.

The use of amisulpride as an anti-emetic is described in WO2011/110854, published on 15 Sep. 2011, which claims priority from British Patent Specification, GB 1004020.2, filed on 11 Mar. 2010. Both of these documents are incorporated into this present specification in their entirety.

In a multi-centre, double-blind, randomised, dose-ranging Phase II trial (conducted by the applicant), amisulpride was given intravenously to adult surgical patients at moderate-to-high risk of suffering PONV (prophylaxis), at doses of 1 mg, 5 mg and 20 mg, with a fourth group receiving placebo. The incidence of PONV was lower in all amisulpride groups, and significantly so in the case of 1 mg (48%, p<0.05) and 5 mg (40%, p<0.01), compared to placebo (69%). This suggests that 5 mg is at or near the bottom of a U-shaped dose response curve when evaluating the incidence of PONV.

In two multi-centre, double-blind, randomised, placebo-controlled Phase III clinical trials conducted by the applicant and involving 626 evaluable, adult surgical patients at moderate-to-high risk of suffering PONV (prophylaxis), administration of amisulpride at 5 mg successfully reduced the incidence of PONV to 48%, compared to 59% with placebo (p<0.01).

In a multi-centre, double-blind, randomised, Phase III trial involving 1147 evaluable adult surgical patients at high risk of suffering PONV (prophylaxis), again conducted by the applicant, amisulpride 5 mg in combination with a standard anti-emetic successfully reduced the incidence of PONV to 42%, compared to 53% with placebo in combination with a standard anti-emetic (p<0.001).

In another clinical trial conducted by the applicant, amisulpride at doses of 5 mg and 10 mg were compared with placebo for the treatment of PONV, in patients who had not received prior prophylaxis. There was no difference between the 5 mg and 10 mg doses in terms of clinical efficacy, suggesting that both doses are at the plateau of the U-shaped dose response curve. Both doses were significantly better than placebo at treating PONV.

Post-operative nausea and/or vomiting is particularly problematic in particular patient groups. In particular, patients with a high (230) BMI, i.e. patients that may be considered as being obese. Moreover, postoperative nausea and/or vomiting can occur in up to 65% of patients after bariatric surgery.

Therefore, there is still a need for the development of novel therapies for the post-operative care of these patient groups.

The listing or discussion of an apparently prior-published document in this specification should not necessarily be taken as an acknowledgement that the document is part of the state of the art or is common general knowledge.

### SUMMARY OF THE INVENTION

The present invention is based on the results of a Phase III study of amisulpride as prophylaxis against PONV in high-risk patients, conducted by the applicant. The Phase III study results include data from two randomized, double-blind, placebo-controlled, multi-centre Phase III trials of amisulpride, either as a prophylaxis, or rescue treatment for PONV.

Post-hoc analysis of the data showed that amisulpride was safe and efficacious for PONV management in three subsets of patients: patients with a high BMI (n=149); patients undergoing bariatric surgery receiving amisulpride as PONV prophylaxis (n=33); and patients undergoing bariatric surgery receiving amisulpride as rescue treatment of established PONV (n=20).

As expected, amisulpride was found to be efficacious in the therapy of PONV, but upon detailed analysis of the data, it was surprisingly found that the relative risk reduction (RRR) of the incidence of PONV in patients with a high (230) BMI was much higher than expected (when compared to RRR of the overall risk of PONV, for example). Therefore, amisulpride is particularly efficacious in the prevention of PONV in patients with a high BMI.

Moreover, it was surprisingly found that, among bariatric surgery patients there was a higher complete response rate (after 24 hours) for patients who received amisulpride prophylaxis or amisulpride rescue treatment compared to placebo. Thus, amisulpride is particularly efficacious in the prevention and treatment of PONV in bariatric surgery patients.

Given bariatric surgery is a weight loss surgery or metabolic surgery, many bariatric surgery patients (although not all) have high BMIs.

As such, according to a first aspect of the invention, there is provided amisulpride for use in the therapy of post-operative nausea and/or vomiting in a patient, wherein the patient:
a) has a BMI of equal to or greater than about 30 (i.e. ≥about 30); and/or
b) is a bariatric surgery patient.

In an alternative first aspect of the invention, there is provided a method of treating or preventing post-operative nausea and/or vomiting in a patient, wherein the patient:
a) has a BMI of equal to or greater than about 30 (i.e. ≥about 30); and/or
b) is a bariatric surgery patient,
   said method comprising administration of an effective amount of a compound of amisulpride to the patient.

In a further alternative first aspect of the invention, there is provided the use of amisulpride, for the manufacture of a medicament for the treatment or prevention of post-operative nausea and/or vomiting in a patient, wherein the patient:
a) has a BMI of equal to or greater than about 30 (i.e. ≥about 30); and/or
b) is a bariatric surgery patient.

### BRIEF DESCRIPTION OF THE DRAWINGS

The following drawings are provided to illustrate various aspects of the present inventive concept and are not intended to limit the scope of the present invention unless specified herein.

FIGS. 1 and 2 show secondary endpoints for amisulpride (5 mg) prophylaxis in patients with high (235) BMI.

FIG. 3 shows complete response data (0 to 24 hours) in bariatric surgery patients who received amisulpride ((5 mg) for prophylaxis and amisulpride (10 mg) for rescue treatment) compared to patients who received placebo.

### DESCRIPTION OF THE INVENTION

Amisulpride has a single chiral centre and two enantiomers exist, i.e. (S−)-amisulpride and (R+)-amisulpride. It may be preferred to use the racemate or an optically active form. Conveniently, (S−)-amisulpride, which is substantially free of the (R+)-enantiomer, may be used. Particularly, the optically active form is (S−)-amisulpride, which is substantially free of the (R+)-amisulpride. It has been reported that almost all of the therapeutic activity is to be found in the (S−)-enantiomer, and therefore use of this enantiomer means that it may be possible to reduce the dose by 50% (e.g., 50%, 60%, 70%, 80%, or 90%, or 50% to 60%, 60% to 70%, 70% to 80%, or 80 to 90%) compared to the racemate.

A racemic mixture of amisulpride means that the amisulpride comprises both the (S−)-amisulpride and the (R+)-enantiomer. For example, the racemic mixture may comprise from 40% to 60% of (S−)-amisulpride and 60% to 40% of the (R+)-enantiomer. A racemic mixture or racemate may comprise about 50% of (S−)-amisulpride and about 50% of the (R+)-enantiomer.

(S−)-amisulpride that is substantially free of the (R+)-enantiomer comprises less than 10%, less than 5%, less than 4%, less than 3%, less than 2%, or less than 1% of (R+)-enantiomer. For example, (S−)-amisulpride that is substantially free of the (R+)-enantiomer comprises less than 2% or less than 1% of (R+)-enantiomer.

As described herein, amisulpride is useful in the therapy of post-operative nausea and/or vomiting in a patient having a BMI of equal to or greater than about 30 (i.e. z about 30).

Body mass index (BMI) is used to determine whether a subject's weight is healthy. The BMI calculation is defined as the subject's weight divided by the square of the subject's height. In adults, an ideal BMI is considered to be in the from 18.5 to 24.9 range. Adults with a BMI of from 25 to 29.9 may be considered to be overweight. A BMI of greater than 30 may be considered to be a “high BMI” and is an indication that a subject might be obese. Accordingly, as described herein, amisulpride may be useful in the therapy of post-operative nausea and/or vomiting in a patient with obesity.

In particular embodiments, amisulpride is useful in the therapy of post-operative nausea and/or vomiting in a patient having a BMI of equal to or greater than about 35 (i.e. ≥about 35).

As used herein, “therapy” means treatment or prevention. Preferably, the amisulpride for use in the invention is used in the prevention of postoperative post-operative nausea and/or vomiting.

As used herein, the term postoperative nausea and/or vomiting (PONV) takes its conventional meaning in the art. It is well understood in the field to mean the occurrence of one or more emetic episodes (vomiting and/or retching) or occurrence of the desire to vomit (nausea), which occurs following a surgical procedure. Retching involves the same physiological mechanisms as vomiting but occurs against a closed glottis. PONV may be defined as nausea and/or vomiting that occurs in the 48-hour period after the end of the surgical procedure. It may be defined as nausea and/or vomiting that occurs in the 24-hour period after the end of the surgical procedure.

As used herein, an “episode of emesis” means the occurrence of an incidence of vomiting and/or an incidence of retching.

As used herein, an “an episode of nausea” means the occurrence of an incidence of nausea. This may be indicated by a patient reporting the desire to vomit or requesting an anti-emetic medication.

In some embodiments, amisulpride according to the present invention is useful in patients undergoing a surgical procedure where postoperative emesis would be potentially dangerous to the patient. For example, an incidence of emesis in these patients could cause hazardous medical complications that are potentially fatal to the patient such as emesis causing sutures to rupture and thereby resulting in a patient bleeding out or allowing a serious infection to take hold.

Further examples of these dangerous/hazardous medical complications caused by postoperative emesis are aspiration into the lungs, suture dehiscence, oesophageal rupture, subcutaneous emphysema, bilateral pneumothoraxes venous hypertension, increased intracranial pressure, or hematomas such as those beneath surgical flaps, vascular anastomoses, and aneurysm clips.

The skilled person will be aware of the surgical procedures in which postoperative emesis would be problematic (or would lead to the complications described above). Examples of these surgical procedures are surgery of the mouth cavity (such as wired jaw surgery or dental surgery), surgery of the ear, nose or throat (ENT) (such as tonsillectomy or thyroidectomy), surgery of the head or face (such as craniotomy, haemorrhagic stroke surgery, ischemic stroke surgery, rhinoplasty, a cosmetic procedure of the face or eye surgery), surgery of the gastrointestinal (GI) tract (such as paraesophageal surgery, anti-reflux surgery, bariatric surgery, gastrectomy, gastric bypass surgery or gastric sleeve surgery), lung surgery (such as a surgical lung biopsy, lobectomy or a wedge resection), abdominal surgery (such as a surgical hernia repair, a total abdominal hysterectomy, abdominoplasty, laparotomy, any surgery involving a large abdominal incision, or an open abdominal aortic aneurysm repair) or bowel surgery.

In particular embodiments, amisulpride according to the present invention may be useful when the patient with a high BMI is undergoing or has undergone bariatric surgery.

As used herein, references to patients will refer to a living subject receiving therapy, including mammalian (e.g. human) patients. Thus, in particular embodiments, the therapy is in a mammal (e.g. a human).

As used herein, a “surgical procedure” takes its conventional meaning in the art. It preferably involves the administration of a general anaesthesia e.g. general inhalation anaesthesia. The procedure may be an elective surgery (open or laparoscopic technique) under general anaesthesia. It is preferably scheduled to last at least one hour from induction of anaesthesia to extubation. Prior to extubation, a wound will be closed.

As used herein “undergoing a surgical procedure” means the time period from about 2 hours preceding the surgical procedure until an episode of PONV in the period of about 24 hours following the surgical procedure (at which stage the therapy ceases to be prevention and is classed as treatment).

In a preferred embodiment, the amisulpride is administered up to 4 hours before the surgical procedure, i.e. to a patient who will undergo surgical procedure. It is preferably administered no later than at the time of wound closure/end of surgery, more preferably at the time of anaesthesia (and more preferably, at the time of induction of the anaesthesia).

As used herein, the term “end of the surgical procedure” takes its conventional meaning in the art and is understood by the skilled person. It usually coincides with a wound closure at the end of the surgery.

As indicated above, bariatric surgery is a surgical procedure in which post-operative nausea and/or vomiting would be particularly problematic. Amisulpride has been found to be particularly effective in the therapy of post-operative nausea and/or vomiting in a bariatric surgery patient. The term “a bariatric surgery patient” herein means a patient who is undergoing, has undergone, or will undergo, bariatric surgery. The skilled person will recognise that the terms “surgical procedure”, “undergoing a surgical procedure”, and “end of the surgical procedure” apply equally to bariatric surgery.

Barlatric surgery (i.e. weight loss surgery or metabolic surgery) includes a variety of surgical procedures performed on patients who need to lose weight, thus often (but not always) the patients will have high BMIs. Examples of bariatric surgery include gastric band surgery, gastric bypass surgery, sleeve gastrectomy, Billopancreatic Diversion with Duodenal Switch and Single Anastomosis Duodeno-Ileal Bypass with Sleeve Gastrectomy. Typically, although not exclusively, bariatric surgery may be used as a treatment for patients who may be considered to be obese or morbidly obese, especially those with a high BMI (i.e. ≥30), particularly those with a BMI ≥35, and more particularly those with a BMI ≥40. Alternatively, the patient undergoing bariatric surgery may not be considered obese or morbidly obese, i.e. has a BMI <30.

Amisulpride may be used in the prevention of post-operative nausea and/or vomiting in patients having a high BMI, i.e. it is administered as described above, but before an incidence of nausea and/or vomiting occurs. In this way, amisulpride is administered with the intention/aim of preventing PONV, i.e. prophylaxis of PONV. Thus, in particular embodiments, the therapy is prophylaxis. Preferably, amisulpride is administered as a single prophylactic dose.

Amisulpride may also be used in the prevention of post-operative nausea and/or vomiting in bariatric surgery patients, such as patients who will or who are undergoing bariatric surgery, i.e. it is administered as described above, but before an incidence of nausea and/or vomiting occurs. In this way, amisulpride is administered with the intention/aim of preventing PONV, i.e. prophylaxis of PONV. Thus, in particular embodiments, the therapy is prophylaxis. Preferably, amisulpride is administered as a single prophylactic dose.

Alternatively, amisulpride according to the present invention may be used as a rescue treatment for PONV (i.e. in patient who had received prior prophylaxis for PONV but who had subsequently suffered from PONV despite the prophylaxis). Thus, in particular embodiments, the patient has already been administered a prophylaxis drug for postoperative nausea and/or vomiting.

As indicated above, in the context of the present invention, a “prophylaxis drug” means a drug that is administered with the intention/aim of preventing PONV. There are many prophylaxis drugs suitable for use in the invention, and these are well known to a person skilled in the art. A particular prophylaxis drug may have been chosen based on a number of different factors, such as age and weight, or whether a person is receiving certain other drugs, for example. Preferably, the prophylaxis drug is an anti-emetic drug that is not amisulpride. More preferably, the prophylaxis drug is not a dopamine-2 (D₂) antagonist.

In some embodiments, the prophylaxis drug is an anti-emetic selected from a 5HT₃-antagonist, a corticosteroid, an anti-histamine (H₁), an anticholinergic, a H₂-antagonist or a NK₁-antagonist.

The 5HT₃-antagonist may be ondansetron, granisetron, palonosetron, tropisetron or dolasetron. It is preferably ondansetron, granisetron or palonosetron. More preferably, it is ondansetron. The corticosterold may be dexamethasone, hydrocortisone, betamethasone, methylprednisolone or prednisolone. It is preferably dexamethasone. The anti-histamine (H₁) may be dimenhydrinate, hydroxazine, diphenhydramine, promethazine, cyclizine or meclizine. The anti-cholinergic may be scopolamine/hycosine. The H₂-antagonist may be famotidine. The NK₁-antagonist may be aprepitant. If a D₂-antagonist is used as the prophylaxis anti-emetic, it may be haloperidol, droperidol or domperidone.

The amisulpride should be administered as soon as is practically possible following a first emetic episode and/or following a first nausea episode (e.g. a first request for anti-emetic medication to treat nausea or a report of the desire to vomit). Preferably, the amisulpride is administered within 1 hour of a first emetic episode and/or within 1 hour of a first nausea episode. More preferably, it is administered within 30 minutes of a first emetic episode and/or within 30 minutes of a first nausea episode. More preferably still, it is administered within 15 minutes of a first emetic episode and/or within 15 minutes of a first nausea episode.

When used as a rescue treatment for PONV (i.e. when the patient has already been administered a prophylaxis drug for PONV) in patients with high (≥30) BMI and/or bariatric surgery patients, it was surprisingly found that amisulpride at a dose of 10 mg was particularly effective. Thus, in particular embodiments, when the patient with high (≥30) BMI and/or the bariatric surgery patient has already been administered a prophylaxis drug for postoperative nausea and/or vomiting, the dose of amisulpride is 7.5 to 15 mg.

Preferably, the dose (i.e. an effective amount) of amisulpride used in rescue treatment for PONV in patients with high (230) BMI and/or bariatric surgery patients comprises 8 to 15 mg amisulpride, more preferably 8.5, 9 or 9.5 to 15 mg. The dose of amisulpride may also be 7.5 to 14.5, 14, 13.5, 13, 12.5, 12, 11.5, 11 or 10.5 mg. Any of the aforementioned limits of the ranges may be combined with each other. Preferably, the dose is 8 to 12 mg, more preferably 9 to 12 mg and most preferably about 10 mg amisulpride. Most preferably, the dose is 10 mg. Preferably, the amisulpride is in the form of a racemic mixture.

Amisulpride for use according to the present invention may be packaged for sale together with accompanying instructions for use. The instructions for use (drug label) may specify that the patient to be treated should have undergone a surgical procedure and that they should be selected from the group of patients who have received prior prophylaxis for PONV that has been unsuccessful (i.e. rescue treatment). They may also preferably specify that the dose of amisulpride is 10 mg.

Alternatively, the instructions for use may specify that the amisulpride is administered with the intention/aim of preventing PONV (i.e. as a prophylaxis drug). In this instance, the instructions for use may specify that the dose of amisulpride is 5 mg.

The instructions for use may specify that the patient has a BMI of equal to or greater than about 30; and/or that they are a bariatric surgery patient.

Amisulpride for use in the present invention is preferably formulated as an intravenous (IV) formulation (and intended for intravenous administration). The amisulpride may be in the form of a salt, hydrate or solvate. Salts include pharmaceutically acceptable salts, for example acid addition salts derived from inorganic or organic acids, such as hydrochlorides, hydrobromides, p-toluenesulphonates, phosphates, sulphates, perchlorates, acetates, trifluoroacetates, propionates, citrates, malonates, succinates, lactates, oxalates, tartrates and benzoates.

Salts may also be formed with bases. Such salts include salts derived from inorganic or organic bases, for example, alkali metal salts such as sodium and potassium salts and alkali earth metal salts such as magnesium and calcium salts, and organic amine salts, such as morpholine, piperidine, dimethylamine and diethylamine salts.

An intravenous formulation of amisulpride for use in the invention may be in the form of a sterile injectable aqueous or non-aqueous (e.g. oleaginous) solution or suspension. The sterile injectable preparation may also be in a sterile injectable solution or suspension in a non-toxic parenterally-acceptable diluent or solvent, for example, a solution in 1,3-butanediol. Among the acceptable vehicles and solvents that may be employed are water, phosphate buffer solution, Ringer's solution and isotonic sodium chloride solution. In addition, sterile, fixed oils may be used as a solvent or suspending medium. For this purpose, any bland fixed oil may be employed, including synthetic mono- or diglycerides. In addition, fatty acids such as oleic acid may be used in the preparation of the intravenous formulation of the invention. Suspensions may be formulated according to the known art using those suitable dispersing or wetting agents and suspending agents.

Aqueous suspensions contain the active ingredient in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Compositions for injection are typically aqueous, and comprise a buffer, e.g. citrate buffer. No other ingredients may be required. The pH of such a composition may be, for example from 4 to 7, e.g. about 5.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known.

An intravenous unit dose of amisulpride suitable for use in the invention is preferably a single injection containing amisulpride. In a preferred embodiment, this could be in the form of a vial of the active agent(s) along with a syringe and needle or a prefilled syringe/needle combination.

Pharmaceutical compositions of amisulpride may be in the form of oil-in-water emulsions. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

In some embodiments, the amisulpride may be in a non-IV injectable formulation. It may be in the form of a solid or liquid formulation, and may be formulated for oral administration. The solid formulations may be in the form of a tablet or capsule, a melt tablet, or in the form of a dispersible powder or granules (that may need to be added to water). Liquid formulations may be in the form of an aqueous or oily suspension or in the form of a syrup, and they may be packaged in a vial.

Amisulpride compositions may also be in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

For topical delivery, transdermal and transmucosal patches, creams, ointments, jellies, solutions or suspensions may be employed.

For sub-lingual delivery, fast dissolving tablet formulations may be used, as well as a number of the presentations described above.

For oral administration, amisulpride may be administered as tablets, capsules or liquids. Oral unit doses of amisulpride may be in the form of one of more tablets, or one or more capsules. The unit doses of amisulpride may be provided in a blister pack.

Amisulpride formulations may contain any number of pharmaceutically acceptable excipients, such as sweeteners and preservatives.

Formulations of amisulpride suitable for use in the invention are described in WO2011/110854.

Preferably, the amisulpride is administered by IV infusion (push), preferably over a time period of from about 20 seconds up to 1 or 2 minutes. In some embodiments, this period may be up to 10 minutes, for example, if the patient has pain on injection or where a higher dose (e.g. 20 mg) is being administered. In a preferred embodiment, the amisulpride is administered over 30 about 1 to 2 minutes, or 1 or 2 minutes. The amisulpride is preferably administered in a single dose.

In some embodiments, no further doses of amisulpride are administered in the 24 hours following the initial dose. In some embodiments, the initial dose according to the invention is followed by at least one other dose within about 24 hours, preferably within about 12 hours, from the first dose.

The dosage regimen utilizing a composition of the invention may be selected in accordance with a variety of factors including type, species, age, weight, sex and/or medical condition of the subject; the severity of the condition to be treated; the route of administration; the renal or hepatic function of the subject; and the particular disclosed compound employed. A physician, clinician or veterinarian of ordinary skill can readily determine the effective amount of each of the active ingredients necessary to prevent, treat or inhibit the progress of the disorder or disease.

In particular embodiments, the dose (i.e. effective amount) of amisulpride comprises from about 1 to about 40 mg amisulpride, more preferably from about 1 to about 20 mg or from about 2.5 to about 20 mg, more preferably from about 5 to about 15 mg. Most preferably, the dose of amisulpride is 10 mg or, even more preferably, 5 mg. An effective amount of amisulpride may also comprise from about 2.5 to about 5 mg, from about 2.5 to about 10 mg, from about 2.5 to about 40 mg, from about 5 to about 20 mg, from about 5 to about 40 mg, from about 1 to about 5 mg or from about 1 to about 10 mg amisulpride.

In particular embodiments, amisulpride is administered as a single daily dose. Preferably, it is administered as a single dose.

In particular embodiments, the amisulpride for use is substantially in the form of a racemate. Alternatively, the amisulpride for use is in the form of (S−)-amisulpride, which is substantially free of the (R+)-enantiomer. If the amisulpride is administered as the S-enantiomer, the dose may be altered accordingly (e.g. it may be halved).

It may be advantageous to administer amisulpride in combination with other classes of drugs which can add additional benefits of efficacy. Preferably, the other classes of drugs are different anti-emetic agents (i.e. an anti-emetic that is not amisulpride). More preferably, the different anti-emetic agent is not a D₂ antagonist. These include, but are not limited to, steroids, most preferably dexamethasone, 5HT₃ antagonists including but not limited to ondansetron, granisetron and palonosetron, and NK₁ antagonists such as aprepitant, netupitant or rolapitant. Preferably, the other anti-emetic agent is ondansetron, granisetron or dexamethasone. Other classes of drugs may be administered via any appropriate routes of administration (e.g., via the route of administration which is typical for that drug, such as oral, intravenous or intramuscular). In some instances, other classes of drugs may be administered within 6 hours from the end of the surgery. In other instances, other classes of drugs may be administered after 6 hours from the end of the surgery.

Typical doses of the different anti-emetic agents listed above are known to a person skilled in the art. For example, ondansetron is typically in a dose of from about 2 to about 20 mg, or about 2 to about 15 mg, or about 10 mg or about 4 mg. For granisetron, the dose is typically from about 1 to about 3 mg (e.g. about 1 mg). For dexamethasone, a typical dose is from about 4 to about 20 mg (e.g. about 4 mg).

Where a use or a method of the invention provides for the administration of more than one drug, they can be administered simultaneous, sequentially or separately. It is not necessary that they are packed together (but this is one embodiment of the invention). It is also not necessary that they are administered at the same time. As used herein, “separate” administration means that the drugs are administered as part of the same overall dosage regimen (which could comprise a number of days), but preferably on the same day. As used herein “simultaneously” means that the drugs are to be taken together or formulated as a single composition. As used herein, “sequentially” means that the drugs are administered at about the same time, and preferably within about 1 hour of each other.

Preferably, the patient has at least 3 risk factors for post-operative emesis, wherein the risk factors are selected from a past history of postoperative nausea and vomiting and/or motion sickness; habitual non-smoking status; being a female; and expected use of post-operative opioid analgesia. More preferably, the patient has all four risk factors. These risk factors may define a patient group for which amisulpride is particularly useful in the therapy of post-operative emesis.

In a particularly preferred embodiment of the invention, amisulpride at a dose of 5 mg is useful in the prevention of postoperative emesis in a patient, preferably wherein the patient is undergoing a surgical procedure where postoperative emesis would be potentially dangerous to the patient, and wherein the patient has at least three risk factors for post-operative emesis, wherein the risk factors are selected from a past history of postoperative nausea and vomiting and/or motion sickness; habitual non-smoking status; being a female; or expected use of post-operative opioid analgesia.

As used herein, the term “about” or “approximately”, when used together with a numeric value (e.g. 5, 10%, ⅓), refers to a range of numeric values that can be less or more than the number. For example, “about 5” refers to a range of numeric values that are 10%, 5%, 2%, or 1% less or more that 5, e.g. a range of 4.5 to 5.5, or 4.75 to 5.25, or 4.9 to 5.1, or 4.95 to 5.05. In some instances, “about 5” refers to a range of numeric values that are 2% or 1% less or more that 5, e.g. a range of 4.9 to 5.1 or 4.95 to 5.05. It is contemplated that, at each instance, such terms may be replaced with the notation “10%”, or the like (or by indicating a variance of a specific amount calculated based on the relevant value). It is also contemplated that, at each instance, such terms may be deleted.

It is noted that aspects of the invention described with respect to one embodiment, may be incorporated in a different embodiment although not specifically described relative thereto. That is, all embodiments and/or features of any embodiment can be combined in any way and/or combination. The applicant reserves the right to change any originally filed claim and/or file any new claim accordingly, including the right to be able to amend any originally filed claim to depend from and/or incorporate any feature of any other claim or claims although not originally claimed in that manner.

These and other objects and/or aspects of the present invention are explained in detail in the specification set forth below.

The following study illustrates the invention.

### Study 1

### Protocol

A randomised, double-blind, placebo-controlled study of amisulpride for IV injection as treatment against post-operative nausea and vomiting in patients having a high BMI (≥30) was conducted. The primary aim of the study was to compare the efficacy of 5 mg and 10 mg amisulpride to placebo as treatment or prevention of PONV, in patients with a high BMI.

The study was performed in adult patients (≥18 years) having a BMI ≥30 who were undergoing elective ambulatory (day-case) or in-patient surgery under general inhalational anaesthesia for an expected duration of at least one hour from induction of anaesthesia to extubation.

Amisulpride at a dose of 5 mg (prophylaxis) or 10 mg (rescue treatment) or matching placebo was given once by slow IV administration, over about 2 minutes.

The primary efficacy variable was the absence or presence of PONV during the 24-hour post-operative period, where PONV was defined as the occurrence of one or more emetic episodes (vomiting and/or retching) or the receipt of one or more doses of rescue anti-emetic medication in the 24 hours after wound closure (prophylaxis study) or dosing (treatment study). Absence of PONV by this definition was termed “Complete Response” (CR). A number of secondary variables were evaluated including the occurrence of emesis (vomiting and/or retching).

### Primary Efficacy Analysis

A comparison of the incidence of CR in the 0-2 and 0-24 hour periods after surgery between the amisulpride group and the placebo group using Pearson's X2 test with Yates's continuity correction at a one-sided significance level of 2.5%. The primary efficacy analysis population was the modified intent-to-treat (mITT) population.

### Secondary Efficacy Analyses

Secondary efficacy variables assessed by incidence (e.g., emesis) were compared between the groups using Pearson's X2 test.

### Results (Extract)

A summary of the data upon which the present invention is based is a follows:

TABLE 1
CR (PONV) data for prophylactic therapy
of patients with a BMI ≥30

| | 0-2 hours | 0-24 hours |
|---|---|---|
| Placebo (234 patients) | 41.9% | 41.9% |
| Amisulpride 5 mg (251 patients) | 53.0% | 53.0% |

TABLE 2
CR (PONV) data for prophylactic therapy
of patients with a BMI ≥35

| | 0-2 hours | 0-24 hours |
|---|---|---|
| Placebo (129 patients) | 55.8% | 32.6% |
| Amisulpride 5 mg (149 patients) | 73.8% (p = 0.002) | 47.7% (p = 0.01) |

TABLE 3
CR (nausea) data for prophylactic
therapy of patients with a high BMI

| | BMI ≥30 | BMI ≥35 |
|---|---|---|
| Placebo | 64.1% (234 patients) | 73.6% (129 patients) |
| Amisulpride 5 mg | 55.0% (251 patients) | 58.4% (149 patients) |

TABLE 4
CR (severe nausea) data for prophylactic
therapy of patients with a high BMI

| | BMI ≥30 | BMI ≥35 |
|---|---|---|
| Placebo | 54.7% (234 patients) | 64.3% (129 patients) |
| Amisulpride 5 mg | 41.8% (251 patients) | 45.0% (149 patients) |

TABLE 5
CR (emesis) data for prophylactic
therapy of patients with a high BMI

| | BMI ≥30 | BMI ≥35 |
|---|---|---|
| Placebo | 18.0% (234 patients) | 18.6% (129 patients) |
| Amisulpride 5 mg | 12.6% (251 patients) | 14.8% (149 patients) |

TABLE 6
CR (PONV) data for rescue treatment of patients with a high BMI

| | BMI ≥30 | BMI ≥35 |
|---|---|---|
| Placebo | 56.0% (234 patients) | 64.3% (129 patients) |
| Amisulpride 10 mg | 46.6% (251 patients) | 51.7% (149 patients) |

TABLE 7
CR (0-24 h) data for prophylaxis^{a}
of PONV in patients with BMI ≥35

| | Amisulpride IV | | | | |
|---|---|---|---|---|---|
| | 5 mg + another | | Placebo + another | | |
| Treatment group | antiemetic | | antiemetic | | P Value |
| All patients | 330/572 | (57.7%) | 268/575 | (46.6%) | <0.001 |
| BMI <35 | 259/423 | (61.2%) | 226/446 | (50.7%) | 0.002 |
| BMI ≥35 | 71/149 | (47.7%) | 42/129 | (32.6%) | 0.011 |

^{a}mITT population

TABLE 8
Treatment-emergent adverse events (≥5%) for amisulpride
(5 mg) prophylaxis in patients with BMI ≥35

| | Amisulpride 5 mg | | Placebo | |
|---|---|---|---|---|
| n (%) | n = 149 | | n = 129 | |
| Number of patients with any adverse event | 79 | (53) | 80 | (62) |
| Nausea | 27 | (18) | 33 | (26) |
| Vomiting | 3 | (2) | 6 | (5) |
| Procedural Pain | 20 | (13) | 20 | (16) |
| Hypertension | 5 | (3) | 6 | (5) |
| Hyperglycemia | 5 | (3) | 6 | (5) |
| Chills | 8 | (5) | 2 | (2) |

TABLE 9
Treatment-emergent adverse events (≥5%) in patients undergoing
bariatric surgery, receiving amisulpride (5 mg) for prophylaxis
or amisulpride (10 mg) for rescue treatment

| | Amisulpride | | Placebo | |
|---|---|---|---|---|
| n (%) | n = 53 | | n = 63 | |
| Number of patients with any adverse event | 30 | (57) | 39 | (62) |
| Nausea | 24 | (45) | 28 | (44) |
| Vomiting | 1 | (2) | 5 | (8) |
| Hypertension | 3 | (6) | 1 | (2) |

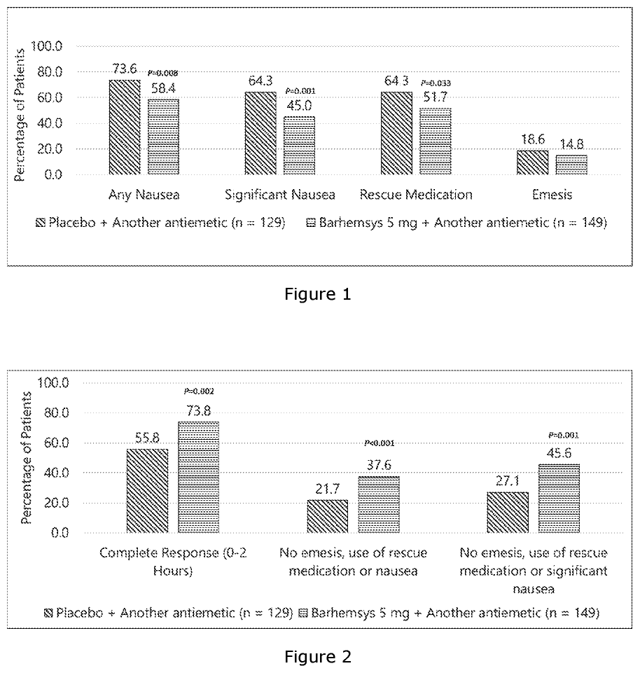

Amisulpride (5 mg) prophylaxis was found to be more effective than placebo in preventing PONV at 24 hours in the overall study population, and particularly in the BMI ≥35 subgroup. As shown in FIGS. 1 and 2, the amisulpride prophylaxis was statistically significantly superior to placebo in all secondary endpoints with the exception of emesis (42/129 for placebo patient group and 71/149 for amisulpride patient group).

The results surprisingly show a 15.10/absolute risk reduction in patients with BMI 235 between patients treated with amisulpride versus placebo. There was a 10.50/absolute risk reduction in patients with a BMI <35 between the amisulpride and placebo treatment groups. The same trends were observed in the 0 to 2 hour time period.

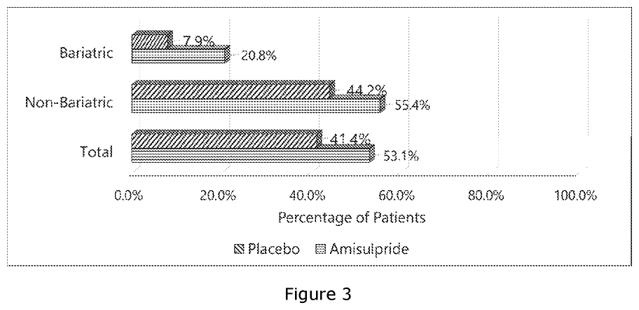

Among bariatric surgery patients, there was a higher complete response rate at 24 hours among those who received amisulpride prophylaxis (18.2% vs. 7.3%, P=0.16) or amisulpride rescue treatment (25.0% vs. 9.1%, P=0.17) compared to patients who received placebo. As shown in FIG. 3, the complete response rate at 24 hours of the combined prophylaxis and rescue treatment bariatric patient subgroup (11/53; 20.8%) was superior compared to the placebo patient subgroup (5/63; 7.9%).

No material difference was observed in the incidence of any treatment-emergent adverse events (TEAE) or serious adverse events (SAE) between the amisulpride and placebo treatment groups, and did not differ from those reported in the larger study populations.

These results indicate that amisulpride prophylaxis was more effective than placebo in preventing PONV for patients with a high BMI. Similarly, there was a higher CR rate among those who received amisulpride rescue treatment compared to placebo.

Moreover, amisulpride was as effective for prophylaxis and rescue treatment of PONV in bariatric surgery patients as in other surgery types.

### CONCLUSION

A single dose of IV amisulpride was demonstrated as safe and effective when administered for the prevention or treatment of PONV in patients with a high BMI. The same conclusion was also reached for the bariatric surgery population.

1. Amisulpride for use in the therapy of post-operative nausea and/or vomiting in a patient, wherein the patient:
a) has a BMI of equal to or greater than about 30; and/or
b) is a bariatric surgery patient.

2. Amisulpride for use according to claim 1, wherein the patient has a BMI of equal to or greater than about 30.

3. Amisulpride for use according to claim 1 or 2 wherein the patient is a bariatric surgery patient.

4. Amisulpride for use according to any preceding claim, wherein the patient has a BMI of equal to or greater than about 35.

5. Amisulpride for use according to any preceding claim, wherein the patient will undergo or is undergoing bariatric surgery, preferably the patient is undergoing bariatric surgery.

6. Amisulpride for use according to any one of claims 1 to 5, wherein the therapy is prophylaxis.

7. Amisulpride for use according to any of claims 1 to 4, wherein the patient has undergone bariatric surgery.

8. Amisulpride for use according to any of claims 1 to 4 or 7, wherein the therapy is treatment, preferably rescue treatment.

9. Amisulpride for use according to any one of claims 1 to 8, wherein the patient has already been administered a prophylaxis drug for postoperative nausea and/or vomiting.

10. Amisulpride for use according to claim 9, wherein the prophylaxis drug is not amisulpride.

11. Amisulpride for use according to claim 9 or claim 10, wherein the prophylaxis drug is not a dopamine-2 (D₂) antagonist.

12. Amisulpride for use according to any one of claims 9 to 11, wherein the prophylaxis drug is an anti-emetic selected from a 5HT₃-antagonist, a corticosteroid, an anti-histamine (H₁), an anti-cholinergic, a H₂-antagonist or a NK₁-antagonist.

13. Amisulpride for use according to any preceding claim, wherein the amisulpride is administered in combination with another anti-emetic, either separately, sequentially or simultaneously.

14. Amisulpride for use according to claim 13, wherein the other anti-emetic is a 5HT₃ antagonist, an NK₁ antagonist or a steroid.

15. Amisulpride for use according to claim 13 or claim 14, wherein the other anti-emetic is dexamethasone, ondansetron, granisetron, palonosetron, aprepitant, netupitant or rolapitant.

16. Amisulpride for use according to any one of claims 1 to 15, wherein the amisulpride is substantially in the form of a racemate.

17. Amisulpride for use according to any one of claims 1 to 16, wherein the amisulpride is in the form of (S−)-amisulpride, which is substantially free of the (R+)-enantiomer.

18. Amisulpride for use according to any one of claims 1 to 17, wherein the amisulpride is administered via the intravenous route.

19. Amisulpride for use according to any one of claims 1 to 18, wherein the amisulpride is administered by IV infusion over from about 1 to about 2 minutes.

20. Amisulpride for use according to any one of claims 1 to 19, wherein the amisulpride is administered in a single dose.

21. Amisulpride for use according to any one of claims 1 to 20, wherein the amisulpride is administered at the time of induction of anaesthesia.

22. Amisulpride for use according to any one of one of claims 1 to 21, wherein the patient is human.

23. Amisulpride for use according to any one of one of claims 1 to 22, wherein the dose of amisulpride is from about 1 to about 40 mg.

24. Amisulpride for use according to any one of claims 1 to 23, wherein the dose of amisulpride is about 1 to about 20 mg.

25. Amisulpride for use according to any one of one of claims 1 to 24, wherein the dose of amisulpride is 5 to 15 mg.

26. Amisulpride for use according to any one of one of claims 1 to 25, wherein the dose of amisulpride is about 7.5 mg to 15 mg, preferably about 10 mg.

27. Amisulpride for use according to any one of one of claims 1 to 25, wherein the dose of amisulpride is about 5 mg.

28. A method of therapy of post-operative nausea and/or vomiting in a patient, wherein the patient:
(a) has a BMI of equal to or greater than about 30 (i.e. ≥about 30); and/or
(b) is a bariatric surgery patient;
said method comprising administration of an effective amount of a compound of amisulpride to the patient.

29. The method according to claim 28, having any of the additional features of claims 2 to 27.

30. Use of amisulpride, for the manufacture of a medicament for the therapy of post-operative nausea and/or vomiting in a patient, wherein the patient:
(a) has a BMI of equal to or greater than about 30 (i.e. ≥about 30); and/or
(b) is a bariatric surgery patient.

31. The use according to claim 30, having any of the additional features of claims 2 to 27.
